(19) United States
(12) Patent Application Publication
(10) Pub. No.: US 2025/0259707 A1
(43) Pub. Date: Aug. 14, 2025

(54) Optimization of Processing Parameters for Top/Middle Down MS/MS

(71) Applicant: DH Technologies Development Pte. Ltd., Singapore (SG)

(72) Inventors: Stephen A. TATE, Barrie (CA); Claudia ALVAREZ, Vaughan (CA); Lyle Lorrence BURTON, Woodbridge (CA)

(21) Appl. No.: 18/856,432

(22) PCT Filed: Mar. 23, 2023
(86) PCT No.: PCT/IB2023/052882

§ 371 Date: Oct. 11, 2024

Related U.S. Application Data
(60) Provisional application No. 63/362,885, filed on Apr. 12, 2022.

(51) Int. Cl. G16B 40/10

(52) U.S. Cl. CPC G16B 40/10

(57) ABSTRACT

In a method for determining if internal product ions are used to provide evidence for a bond of a polymeric compound, two or more theoretical product ions resulting from the cleavage of at least one bond of the sequence of the compound are calculated. A product ion spectrum is searched for the theoretical product ions. The theoretical internal product ions are calculated and the spectrum is searched for the theoretical internal product ions if one or more theoretical product ions of the theoretical product ions match a product ion of the spectrum. In another embodiment, a mass tolerance is automatically determined for comparing theoretical mass peaks to mass peaks of an experimental mass spectrum using the subset of product ions most likely to be found for the fragmentation method used. In another embodiment, charge filtering is used to find an experimental product ion of a compound.

### RELATED APPLICATIONS

This application claims the benefit of U.S. Provisional Patent Application Ser. No. 63/362,885, filed on Apr. 12, 2022, the content of which is incorporated by reference herein in its entirety.

### FIELD

The teachings herein relate to three different methods for improving automatic peak finding and charge state calling in spectra produced for polymeric compounds.

### INTRODUCTION

### Peak Finding and Charge State Calling Limitations

Top and middle-down fragmentation of a protein or other polymeric compound generates highly complex spectra, especially when electron-capture dissociation (ECD) fragmentation is used. The fragments typically cover a large range of charge states and there can be a significant overlap of different isotope clusters.

In order to confirm (or not) that the data matches the expected sequence, theoretical fragments need to be compared to the experimental data. The complexity makes automated peak finding and (especially) charge state calling difficult.

FIG. 2 is an exemplary zoomed-in spectral plot 200 of an experimental product ion spectrum where charge state calling is confounded by the complexity of the spectrum, in accordance with various embodiments. In FIG. 2, experimental product ion peaks 210 of the experimental spectrum are shown in comparison with theoretical isotope pattern 220. Theoretical isotope pattern 220 is for a putative +12 fragment of the protein.

There is clearly evidence for the +12 fragment of the protein among product ion peaks 210 of the experimental spectrum. However, the peak finding algorithm used was not able to determine the charge state due to a confounding +3 fragment represented by product ion peaks 211 of the experimental spectrum. FIG. 2 shows that users must often spend considerable time manually reviewing the data produced from top and middle-down fragmentation of a protein or other polymeric compound.

As a result, additional systems and methods are needed to improve automatic peak finding and charge state calling in spectra produced for polymeric compounds.

### LC-MS and LC-MS/MS Background

Mass spectrometry (MS) is an analytical technique for the detection and quantitation of chemical compounds based on the analysis of mass-to-charge ratios (m/z) of ions formed from those compounds. The combination of mass spectrometry (MS) and liquid chromatography (LC) is an important analytical tool for the identification and quantitation of compounds within a mixture. Generally, in liquid chromatography, a fluid sample under analysis is passed through a column filled with a chemically-treated solid adsorbent material (typically in the form of small solid particles, e.g., silica). Due to slightly different interactions of components of the mixture with the solid adsorbent material (typically referred to as the stationary phase), the different components can have different transit (elution) times through the packed column, resulting in separation of the various components.

Note that the terms “mass” and “m/z” are used interchangeably herein. One of ordinary skill in the art understands that a mass can be found from an m/z by multiplying the m/z by the charge. Similarly, the m/z can be found from a mass by dividing the mass by the charge.

In LC-MS, the effluent exiting the LC column can be continuously subjected to MS analysis. The data from this analysis can be processed to generate an extracted ion chromatogram (XIC), which can depict detected ion intensity (a measure of the number of detected ions of one or more particular analytes) as a function of retention time.

In MS analysis, an MS or precursor ion scan is performed at each interval of the separation for a mass range that includes the precursor ion. An MS scan includes the selection of a precursor ion or precursor ion range and mass analysis of the precursor ion or precursor ion range.

In some cases, the LC effluent can be subjected to tandem mass spectrometry (or mass spectrometry/mass spectrometry MS/MS) for the identification of product ions corresponding to the peaks in the XIC. For example, the precursor ions can be selected based on their mass/charge ratio to be subjected to subsequent stages of mass analysis. For example, the selected precursor ions can be fragmented (e.g., via collision-induced dissociation), and the fragmented ions (product ions) can be analyzed via a subsequent stage of mass spectrometry.

### Fragmentation Techniques Background

Electron-based dissociation (ExD), ultraviolet photodissociation (UVPD), infrared photodissociation (IRMPD), and collision-induced dissociation (CID) are often used as fragmentation techniques for tandem mass spectrometry (MS/MS). CID is the most conventional technique for dissociation in tandem mass spectrometers. ExD can include, but is not limited to, electron-induced dissociation (EID), electron impact excitation in organics (EIEIO), electron capture dissociation (ECD), or electron transfer dissociation (ETD).

### Tandem Mass Spectrometry or MS/MS Background

Tandem mass spectrometry or MS/MS involves ionization of one or more compounds of interest from a sample, selection of one or more precursor ions of the one or more compounds, fragmentation of the one or more precursor ions into product ions, and mass analysis of the product ions.

Tandem mass spectrometry can provide both qualitative and quantitative information. The product ion spectrum can be used to identify a molecule of interest. The intensity of one or more product ions can be used to quantitate the amount of the compound present in a sample.

A large number of different types of experimental methods or workflows can be performed using a tandem mass spectrometer. These workflows can include, but are not limited to, targeted acquisition, information dependent acquisition (IDA) or data dependent acquisition (DDA), and data independent acquisition (DIA).

In a targeted acquisition method, one or more transitions of a precursor ion to a product ion are predefined for a compound of interest. As a sample is being introduced into the tandem mass spectrometer, the one or more transitions are interrogated during each time period or cycle of a plurality of time periods or cycles. In other words, the mass spectrometer selects and fragments the precursor ion of each transition and performs a targeted mass analysis for the product ion of the transition. As a result, a chromatogram (the variation of the intensity with retention time) is produced for each transition. Targeted acquisition methods include, but are not limited to, multiple reaction monitoring (MRM) and selected reaction monitoring (SRM).

MRM experiments are typically performed using “low resolution” instruments that include, but are not limited to, triple quadrupole (QqQ) or quadrupole linear ion trap (QqLIT) devices. With the advent of “high resolution” instruments, there was a desire to collect MS and MS/MS using workflows that are similar to QqQ/QqLIT systems. High-resolution instruments include, but are not limited to, quadrupole time-of-flight (QqTOF) or orbitrap devices. These high-resolution instruments also provide new functionality.

MRM on QqQ/QqLIT systems is the standard mass spectrometric technique of choice for targeted quantification in all application areas, due to its ability to provide the highest specificity and sensitivity for the detection of specific components in complex mixtures. However, the speed and sensitivity of today's accurate mass systems have enabled a new quantification strategy with similar performance characteristics. In this strategy (termed MRM high resolution (MRM-HR) or parallel reaction monitoring (PRM)), looped MS/MS spectra are collected at high-resolution with short accumulation times, and then fragment ions (product ions) are extracted post-acquisition to generate MRM-like peaks for integration and quantification. With instrumentation like the TRIPLETOF® Systems of AB SCIEX™, this targeted technique is sensitive and fast enough to enable quantitative performance similar to higher-end triple quadrupole instruments, with full fragmentation data measured at high resolution and high mass accuracy.

In other words, in methods such as MRM-HR, a high-resolution precursor ion mass spectrum is obtained, one or more precursor ions are selected and fragmented, and a high-resolution full product ion spectrum is obtained for each selected precursor ion. A full product ion spectrum is collected for each selected precursor ion but a product ion mass of interest can be specified and everything other than the mass window of the product ion mass of interest can be discarded.

In an IDA (or DDA) method, a user can specify criteria for collecting mass spectra of product ions while a sample is being introduced into the tandem mass spectrometer. For example, in an IDA method a precursor ion or mass spectrometry (MS) survey scan is performed to generate a precursor ion peak list. The user can select criteria to filter the peak list for a subset of the precursor ions on the peak list. The survey scan and peak list are periodically refreshed or updated, and MS/MS is then performed on each precursor ion of the subset of precursor ions. A product ion spectrum is produced for each precursor ion. MS/MS is repeatedly performed on the precursor ions of the subset of precursor ions as the sample is being introduced into the tandem mass spectrometer.

In proteomics and many other applications, however, the complexity and dynamic range of compounds is very large. This poses challenges for traditional targeted and IDA methods, requiring very high-speed MS/MS acquisition to deeply interrogate the sample in order to both identify and quantify a broad range of analytes.

As a result, DIA methods, the third broad category of tandem mass spectrometry, were developed. These DIA methods have been used to increase the reproducibility and comprehensiveness of data collection from complex samples. DIA methods can also be called non-specific fragmentation methods. In a DIA method the actions of the tandem mass spectrometer are not varied among MS/MS scans based on data acquired in a previous precursor or survey scan. Instead, a precursor ion mass range is selected. A precursor ion mass selection window is then stepped across the precursor ion mass range. All precursor ions in the precursor ion mass selection window are fragmented and all of the product ions of all of the precursor ions in the precursor ion mass selection window are mass analyzed.

The precursor ion mass selection window used to scan the mass range can be narrow so that the likelihood of multiple precursors within the window is small. This type of DIA method is called, for example, MS/MS^{ALL}. In an MS/MS^{ALL} method, a precursor ion mass selection window of about 1 Da is scanned or stepped across an entire mass range. A product ion spectrum is produced for each 1 Da precursor mass window. The time it takes to analyze or scan the entire mass range once is referred to as one scan cycle. Scanning a narrow precursor ion mass selection window across a wide precursor ion mass range during each cycle, however, can take a long time and is not practical for some instruments and experiments.

As a result, a larger precursor ion mass selection window, or selection window with a greater width, is stepped across the entire precursor mass range. This type of DIA method is called, for example, SWATH acquisition. In a SWATH acquisition, the precursor ion mass selection window stepped across the precursor mass range in each cycle may have a width of 5-25 Da, or even larger. Like the MS/MS^{ALL} method, all of the precursor ions in each precursor ion mass selection window are fragmented, and all of the product ions of all of the precursor ions in each mass selection window are mass analyzed. However, because a wider precursor ion mass selection window is used, the cycle time can be significantly reduced in comparison to the cycle time of the MS/MS^{ALL} method.

U.S. Pat. No. 8,809,770 describes how SWATH acquisition can be used to provide quantitative and qualitative information about the precursor ions of compounds of interest. In particular, the product ions found from fragmenting a precursor ion mass selection window are compared to a database of known product ions of compounds of interest. In addition, ion traces or extracted ion chromatograms (XICs) of the product ions found from fragmenting a precursor ion mass selection window are analyzed to provide quantitative and qualitative information.

However, identifying compounds of interest in a sample analyzed using SWATH acquisition, for example, can be difficult. It can be difficult because either there is no precursor ion information provided with a precursor ion mass selection window to help determine the precursor ion that produces each product ion, or the precursor ion information provided is from a mass spectrometry (MS) observation that has a low sensitivity. In addition, because there is little or no specific precursor ion information provided with a precursor ion mass selection window, it is also difficult to determine if a product ion is convolved with or includes contributions from multiple precursor ions within the precursor ion mass selection window.

As a result, a method of scanning the precursor ion mass selection windows in SWATH acquisition, called scanning SWATH, was developed. Essentially, in scanning SWATH, a precursor ion mass selection window is scanned across a mass range so that successive windows have large areas of overlap and small areas of non-overlap. This scanning makes the resulting product ions a function of the scanned precursor ion mass selection windows. This additional information, in turn, can be used to identify the one or more precursor ions responsible for each product ion.

Scanning SWATH has been described in International Publication No. WO 2019/171459 A2 (hereinafter “the '459 Application”). In the '459 Application, a precursor ion mass selection window or precursor ion mass selection window of 25 Da is scanned with time such that the range of the precursor ion mass selection window changes with time. The timing at which product ions are detected is then correlated to the timing of the precursor ion mass selection window in which their precursor ions were transmitted.

The correlation is done by first plotting the mass-to-charge ratio (m/z) of each product ion detected as a function of the precursor ion m/z values transmitted by the quadrupole mass filter. Since the precursor ion mass selection window is scanned over time, the precursor ion m/z values transmitted by the quadrupole mass filter can also be thought of as times. The start and end times at which a particular product ion is detected are correlated to the start and end times at which its precursor is transmitted from the quadrupole. As a result, the start and end times of the product ion signals are used to determine the start and end times of their corresponding precursor ions.

### SUMMARY

The teachings herein relate to three different methods for improving automatic peak finding and charge state calling in spectra produced for polymeric compounds. A first method determines if internal product ions are used to provide evidence for a bond of a polymeric compound. A second method automatically determines a mass tolerance for comparing theoretical mass peaks to mass peaks of an experimental mass spectrum. A third method finds an experimental product ion of a compound using charge filtering.

The systems and methods herein can be performed in conjunction with a processor, controller, or computer system, such as the computer system of FIG. 1.

A system, method, and computer program product are disclosed for determining if internal product ions are used to provide evidence for a bond of a polymeric compound. A sequence and at least one product ion spectrum are received for a polymeric compound. Two or more theoretical product ions resulting from the cleavage of at least one bond of the sequence are calculated. The at least one spectrum is searched for the two or more theoretical product ions. If one or more theoretical product ions of the two or more theoretical product ions match a product ion of the at least one spectrum, theoretical internal product ions are calculated for the one or more theoretical product ions and the at least one spectrum is searched for theoretical internal product ions of the one or more theoretical product ions.

A system, method, and computer program product are disclosed for automatically determining a mass tolerance for comparing theoretical mass peaks to mass peaks of an experimental mass spectrum. At least one product ion spectrum for a compound and a most likely product ion type for the fragmentation method used to produce the at least one spectrum are received. A plurality of theoretical product ions is calculated for the compound. A subset of theoretical product ions of the plurality of theoretical product ions that are of the most likely product ion type is determined. Each product ion of the subset of theoretical product ions is compared to one or more product ions of the at least one spectrum using a predetermined mass tolerance. If a theoretical product ion of the subset matches an experimental product of the at least one spectrum, a mass error is calculated for the matching theoretical and experimental product ions. A plurality of matching theoretical product ions of the subset and a corresponding plurality of mass errors are produced. A mass tolerance for comparing the plurality of theoretical product ions to the at least one spectrum is calculated from a statistical central tendency measure of the plurality of mass errors.

A system, method, and computer program product are disclosed for finding an experimental product ion of a compound using charge states. At least one product ion spectrum of a compound is received. For at least one theoretical product ion of the compound, a plurality of theoretical isotope patterns corresponding to different possible charge states of the theoretical product ion is calculated.

Each pattern of the plurality of theoretical isotope patterns is convolved with product ion peaks of the at least one product ion spectrum. If a theoretical isotope pattern of the plurality of theoretical isotope patterns aligns with a number of peaks of a product ion of the at least one spectrum that is greater than a predetermined threshold number, the product ion matches the at least one theoretical product ion of the compound.

These and other features of the applicant's teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings may be implemented.

FIG. 2 is an exemplary zoomed-in spectral plot of an experimental product ion spectrum where charge state calling is confounded by the complexity of the spectrum, in accordance with various embodiments.

FIG. 3 is a schematic diagram of a system for determining if internal product ions are used to provide evidence for a bond of a polymeric compound, in accordance with various embodiments.

FIG. 4 is an exemplary flowchart showing a method for determining if internal product ions are used to provide evidence for a bond of a polymeric compound, in accordance with various embodiments.

FIG. 5 is a schematic diagram of a system that includes one or more distinct software modules and that performs a method for determining if internal product ions are used to provide evidence for a bond of a polymeric compound, in accordance with various embodiments.

FIG. 6 is a schematic diagram of a system for automatically determining a mass tolerance for comparing theoretical mass peaks to mass peaks of an experimental mass spectrum, in accordance with various embodiments.

FIG. 7 is an exemplary flowchart showing a method for automatically determining a mass tolerance for comparing theoretical mass peaks to mass peaks of an experimental mass spectrum, in accordance with various embodiments.

FIG. 8 is a schematic diagram of a system that includes one or more distinct software modules and that performs a method for automatically determining a mass tolerance for comparing theoretical mass peaks to mass peaks of an experimental mass spectrum, in accordance with various embodiments.

FIG. 9 is a schematic diagram of a system for finding an experimental product ion of a compound using charge states, in accordance with various embodiments.

FIG. 10 is an exemplary flowchart showing a method for finding an experimental product ion of a compound using charge states, in accordance with various embodiments.

FIG. 11 is a schematic diagram of a system that includes one or more distinct software modules and that performs a method for finding an experimental product ion of a compound using charge states, in accordance with various embodiments.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DESCRIPTION OF VARIOUS EMBODIMENTS

### Computer-Implemented System

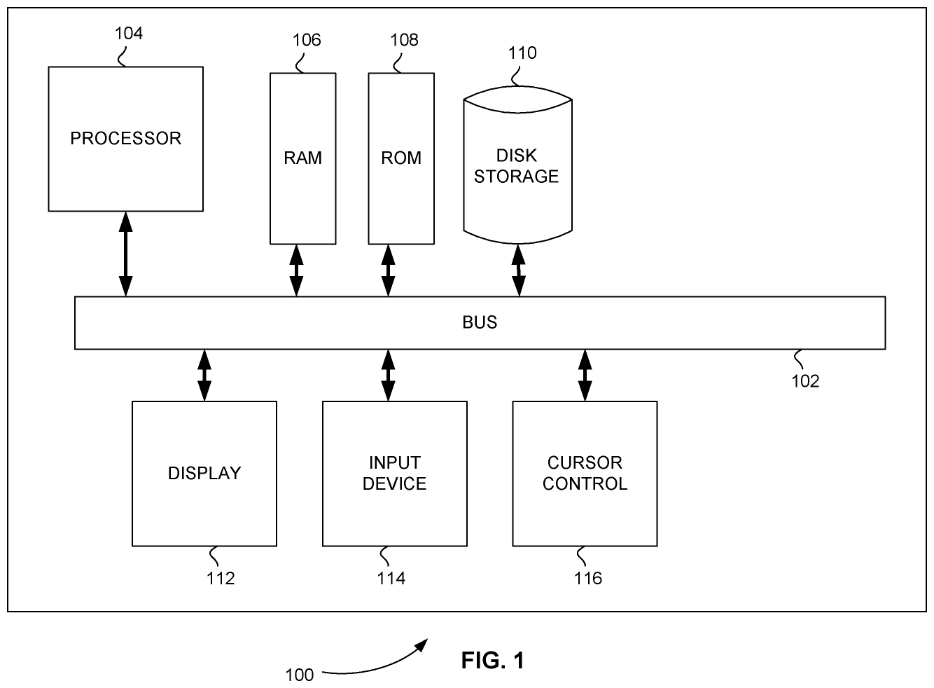

FIG. 1 is a block diagram that illustrates a computer system 100, upon which embodiments of the present teachings may be implemented. Computer system 100 includes a bus 102 or other communication mechanism for communicating information, and a processor 104 coupled with bus 102 for processing information. Computer system 100 also includes a memory 106, which can be a random-access memory (RAM) or other dynamic storage device, coupled to bus 102 for storing instructions to be executed by processor 104. Memory 106 also may be used for storing temporary variables or other intermediate information during execution of instructions to be executed by processor 104. Computer system 100 further includes a read only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112.

A computer system 100 can perform the present teachings. Consistent with certain implementations of the present teachings, results are provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 causes processor 104 to perform the process described herein.

Alternatively, hard-wired circuitry may be used in place of or in combination with software instructions to implement the present teachings. For example, the present teachings may also be implemented with programmable artificial intelligence (AI) chips with only the encoder neural network programmed—to allow for performance and decreased cost. Thus, implementations of the present teachings are not limited to any specific combination of hardware circuitry and software.

The term “computer-readable medium” or “computer program product” as used herein refers to any media that participates in providing instructions to processor 104 for execution. The terms “computer-readable medium” and “computer program product” are used interchangeably throughout this written description. Such a medium may take many forms, including but not limited to, non-volatile media and volatile media. Non-volatile media includes, for example, optical or magnetic disks, such as storage device 110. Volatile media includes dynamic memory, such as memory 106.

Common forms of computer-readable media include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

### Improvements for Automated Peak Finding

As described above, top and middle-down fragmentation of a protein or other polymeric compound generates highly complex spectra. In order to confirm (or not) that the data matches the expected sequence, theoretical fragments need to be compared to the experimental data.

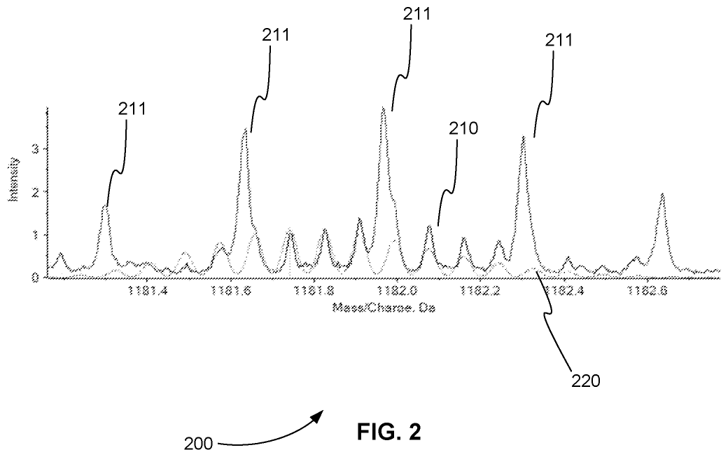

The complexity of the spectra, however, makes automated peak finding and (especially) charge state calling difficult. FIG. 2 shows that users must often spend considerable time manually reviewing the data produced from top and middle-down fragmentation of a protein or other polymeric compound.

As a result, additional systems and methods are needed to improve automatic peak finding and charge state calling in spectra produced for polymeric compounds.

In various embodiments, various improvements are provided to automatic peak finding and charge state calling. In particular, specific systems and methods used in automatic peak finding and charge state calling are optimized.

In one embodiment, for example, systems and methods for determining whether or not internal product ions should be used as evidence for a bond are optimized. Internal fragments (i.e., where two peptide bonds are broken) can be important for maximizing sequence/bond coverage. However, a great many theoretical possibilities exist so this not only takes longer to process, but there are more false positives as a result and more data to review. In this embodiment, false positives are reduced by only considering possibilities where there is evidence from the standard b/y/c′/z^{•} fragments for one of the broken bond positions. This limitation on the use of internal fragments is applied after first round standard fragments (b/y/c′/z^{•}, i.e. a single peptide bond breakage) have been generated and matched so that second round internal fragments do not need to be generated for unmatched bonds, hence speeding the process (i.e., rather than just filtered at the end).

In another embodiment, systems and methods use the most confident matches for the most likely fragment types (i.e., c′/z^{•} for ECD fragmentation) to automatically recalibrate the product ions of the experimental spectrum. When this is not done, a large mass tolerance can be used and more false positives are produced. After recalibration, the mass-to-charge ratio (m/z) errors for the confident matches can be used to set an automatic mass tolerance for other matches. Conventionally, this is set manually.

In another embodiment, a charge filtering algorithm is used to determine charge states more accurately than the approach commonly used, at least for lower molecular weight peptides. Charge filtering is a technique where the charge state for an m/z peak is determined by convolving the experimental spectrum with a theoretical isotope pattern for a number of different possible charge states. For the correct charge state, the peaks in the experimental and theoretical pattern will all be in alignment and the response will be highest; for incorrect charge states fewer peaks will be in alignment and the response is lower. So, the charge state which gives the largest response is taken as the best estimate of the charge.

Charge filtering has not been used before for MS/MS. Charge filtering is slower than conventional techniques, so, in various embodiments, it is only applied to complex or convolved regions (as in FIG. 2) and not when the conventional technique finds a match.

### System for Determining if Internal Product Ions are Used

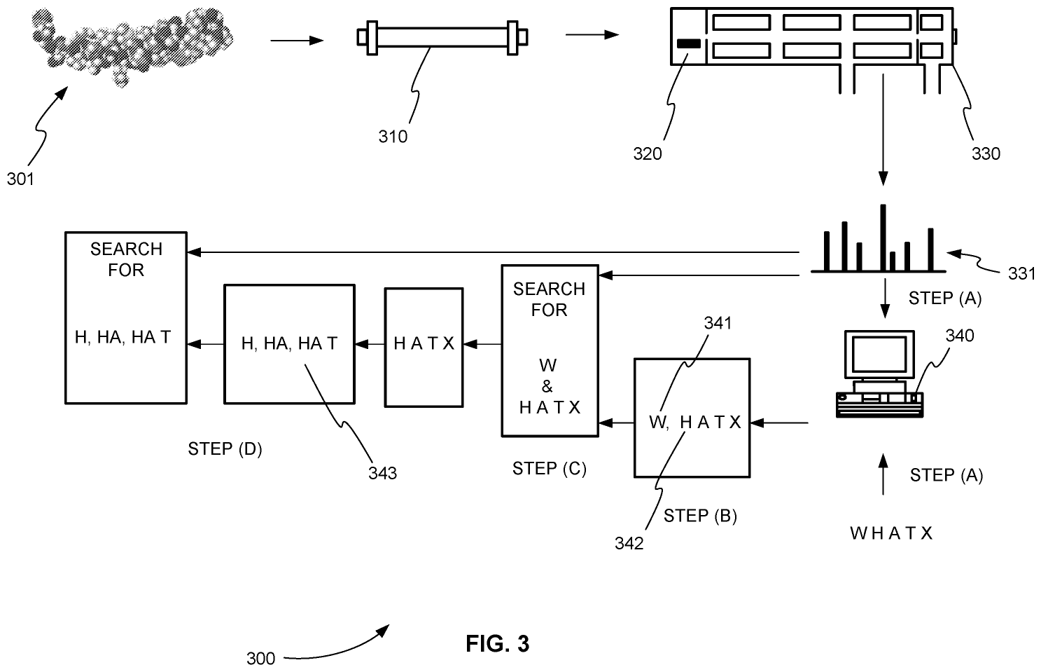

FIG. 3 is a schematic diagram 300 of a system for determining if internal product ions are used to provide evidence for a bond of a polymeric compound, in accordance with various embodiments. The system includes processor 340. Processor 340 can be, but is not limited to, a controller, a computer, a microprocessor, the computer system of FIG. 1, or any device capable of analyzing data. Processor 340 can also be any device capable of sending and receiving control signals and data.

In a step (A), processor 340 receives a sequence and at least one product ion spectrum 331 for a polymeric compound 301 and the expected sequence. In step (B), processor 340 calculates two or more theoretical product ions 341 and 342 resulting from the cleavage of at least one bond of the sequence.

In step (C), processor 340 searches spectrum 331 for the two or more theoretical product ions 341 and 342

In step (D), if one or more theoretical product ions of the two or more theoretical product ions 341 and 342 match a product ion of spectrum 331, processor 340 calculates theoretical internal product ions for the one or more theoretical product ions and searches spectrum 331 for theoretical internal product ions of the one or more theoretical product ions.

In various embodiments, if one or more theoretical product ions of the two or more theoretical product ions 341 and 342 match a product ion of spectrum 331, processor 340 calculates theoretical internal product ions for the two or more theoretical product ions and searches spectrum 331 for theoretical internal product ions of the two or more theoretical product ions

In FIG. 3, for example, theoretical product ion 342 is found to match a product ion of spectrum 331. As a result, theoretical internal product ions 343 are calculated for theoretical product ion 342. Spectrum 331 is then searched for theoretical internal product ions 343.

In various embodiments, further in step (D), if the one or more theoretical product ions of the two or more theoretical product ions 341 and 342 are b, y, c′, or z^{•} product ions and match a product ion of spectrum 331, processor 340 calculates theoretical internal product ions for the one or more b, y, c′, or z^{•} theoretical product ions and searches spectrum 331 for theoretical internal product ions of the one or more b, y, c′, or z^{•} theoretical product ions.

In various embodiments, further in step (D), if all of the two or more theoretical product ions 341 and 342 match a product ion of the at least one spectrum, processor 340 calculates theoretical internal product ions for both theoretical product ions 341 and 342 and searching the at least one spectrum for theoretical internal product ions of both theoretical product ions 341 and 342.

In various embodiments, the system of FIG. 3 further includes mass spectrometer 330 that measures mass spectrum 331 and sends mass spectrum 331 to processor 340. Ion source device 320 of mass spectrometer 330 ionizes separated fragments of compound 301 or only compound 301, producing an ion beam. Ion source device 320 is controlled by processor 340, for example. Ion source device 320 is shown as a component of mass spectrometer 330. In various alternative embodiments, ion source device 320 is a separate device. Ion source device 320 can be, but is not limited to, an electrospray ion source (ESI) device or a chemical ionization (CI) source device such as an atmospheric pressure chemical ionization source (APCI) device or an atmospheric pressure photoionization (APPI) source device.

Mass spectrometer 330 mass analyzes precursor ions of compound 301 or selects and fragments compound 301 and mass analyzes product ions of compound 301 from the ion beam at a plurality of different times. Mass spectrum 331 is produced for compound 301. Mass spectrometer 330 is controlled by processor 340, for example.

In the system of FIG. 3, mass spectrometer 330 is shown as a triple quadrupole device. One of ordinary skill in the art can appreciate that any component of mass spectrometer 330 can include other types of mass spectrometry devices including, but not limited to, ion traps, orbitraps, time-of-flight (TOF) devices, ion mobility devices, or Fourier transform ion cyclotron resonance (FT-ICR) devices.

In various embodiments, the system of FIG. 3 further includes additional device 310 that affects compound 301, providing the at least one additional dimension. As shown in FIG. 3, additional device 310 is an LC device and the at least one additional dimension or spectral data provided is retention time. In various alternative embodiments, additional device 310 can be, but is not limited to, a gas chromatography (GC) device, capillary electrophoresis (CE) device, an ion mobility spectrometry (IMS) device, or a differential mobility spectrometry (DMS) device. In still further embodiments, additional device 310 is not used and the at least one additional dimension or spectral data provided is precursor ion m/z and is provided by mass spectrometer 330 operating in a precursor ion scanning mode.

### Method for Determining if Internal Product Ions are Used

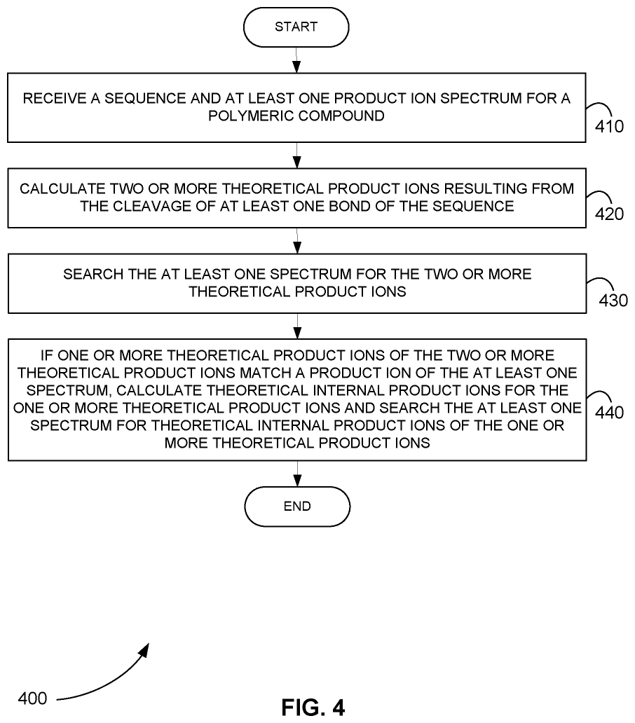

FIG. 4 is an exemplary flowchart showing a method 400 for determining if internal product ions are used to provide evidence for a bond of a polymeric compound, in accordance with various embodiments.

In step 410 of method 400, a sequence and at least one product ion spectrum are received for a polymeric compound.

In step 420, two or more theoretical product ions resulting from the cleavage of at least one bond of the sequence are calculated.

In step 430, the at least one spectrum is searched for the two or more theoretical product ions.

In step 440, if one or more theoretical product ions of the two or more theoretical product ions match a product ion of the at least one spectrum, theoretical internal product ions are calculated for the one or more theoretical product ions and the at least one spectrum is searched for theoretical internal product ions of the one or more theoretical product ions.

### Computer Program Product for Determining if Internal Product Ions are Used

In various embodiments, a computer program product includes a non-transitory tangible computer-readable storage medium whose contents include a program with instructions being executed on a processor so as to perform a method for determining if internal product ions are used to provide evidence for a bond of a polymeric compound. This method is performed by a system that includes one or more distinct software modules.

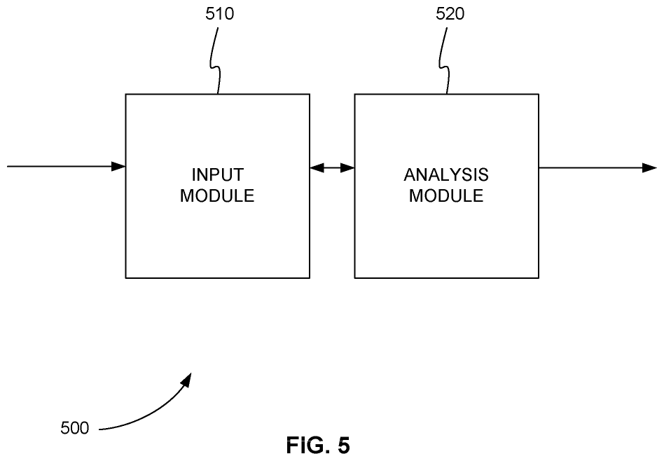

FIG. 5 is a schematic diagram of a system 500 that includes one or more distinct software modules and that performs a method for determining if internal product ions are used to provide evidence for a bond of a polymeric compound, in accordance with various embodiments. System 500 includes input module 510 and analysis module 520.

Input module 510 receives a sequence and at least one product ion spectrum for a polymeric compound.

Analysis module 520 calculates two or more theoretical product ions resulting from the cleavage of at least one bond of the sequence. Analysis module 520 searches the at least one spectrum for the two or more theoretical product ions. If one or more theoretical product ions of the two or more theoretical product ions match a product ion of the at least one spectrum, analysis module 520 calculates theoretical internal product ions for the one or more theoretical product ions and searches the at least one spectrum for theoretical internal product ions of the one or more theoretical product ions.

### System for Automatically Determining a Mass Tolerance

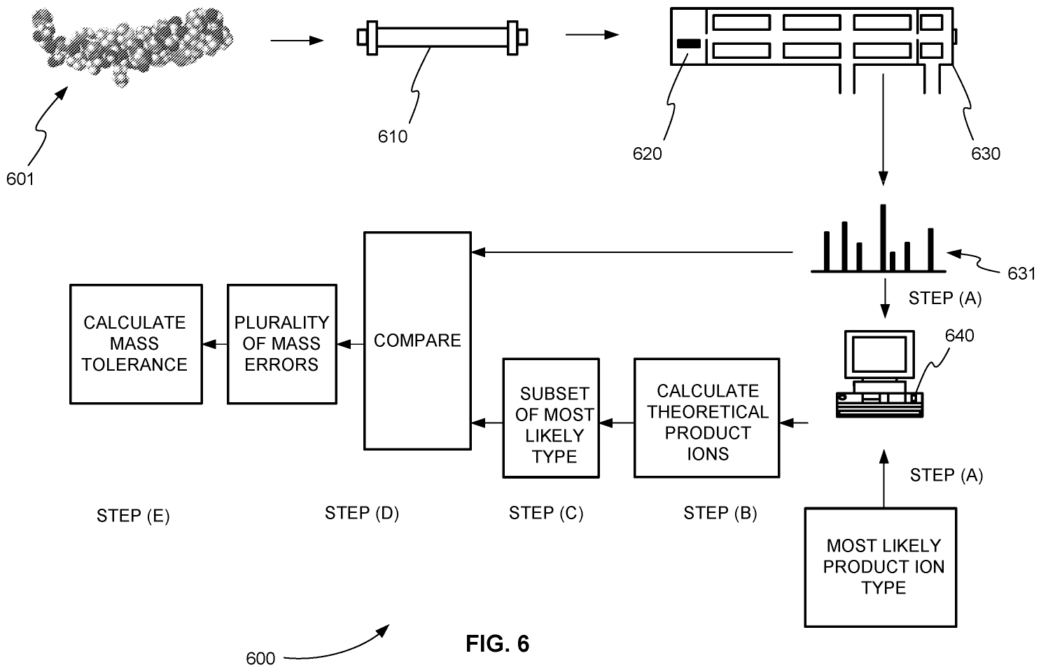

FIG. 6 is a schematic diagram 600 of a system for automatically determining a mass tolerance for comparing theoretical mass peaks to mass peaks of an experimental mass spectrum, in accordance with various embodiments. The system includes processor 640. Processor 640 can be, but is not limited to, a controller, a computer, a microprocessor, the computer system of FIG. 1, or any device capable of analyzing data. Processor 640 can also be any device capable of sending and receiving control signals and data.

In a step (A), processor 640 receives at least one product ion spectrum 631 for a compound 601 and a most likely product ion type for the fragmentation method used to produce the at least one spectrum. In step (B), processor 640 calculates a plurality of theoretical product ions for the compound.

In step (C), processor 640 determines a subset of theoretical product ions of the plurality of theoretical product ions that are of the most likely product ion type.

In step (D), processor 640 compares each product ion of the subset of theoretical product ions to one or more product ions of spectrum 631 and, if a theoretical product ion of the subset matches an experimental product of spectrum 631, calculates a mass error for the matching theoretical and experimental product ions. A plurality of matching theoretical product ions of the subset and a corresponding plurality of mass errors are produced.

In various embodiments, an isotope or isotope pattern can be used to compare each product ion of the subset of theoretical product ions to one or more product ions of spectrum 631. In various embodiments, a predetermined mass tolerance can be used to compare each product ion of the subset of theoretical product ions to one or more product ions of spectrum 631.

In step (E), processor 640 calculates a mass tolerance for comparing the plurality of theoretical product ions to the at least one spectrum from a statistical central tendency measure of the plurality of mass errors.

In various embodiments, compound 601 includes a polymeric compound and the plurality of theoretical product ions are calculated for one or more bonds of the polymeric compound.

In various embodiments, the statistical central tendency measure includes one of the mean, mode, or median.

In various embodiments, the system of FIG. 6 further includes mass spectrometer 630 that measures mass spectrum 631 and sends mass spectrum 631 to processor 630. Ion source device 620 of mass spectrometer 630 ionizes separated fragments of compound 601 or only compound 601, producing an ion beam. Ion source device 620 is controlled by processor 640, for example. Ion source device 620 is shown as a component of mass spectrometer 630. In various alternative embodiments, ion source device 620 is a separate device. Ion source device 620 can be, but is not limited to, an ESI device or a CI source device such as an APCI device or an APPI source device.

Mass spectrometer 630 mass analyzes product ions of compound 601 or selects and fragments compound 601 and mass analyzes product ions of compound 601 from the ion beam at a plurality of different times. Mass spectrum 631 is produced for compound 601. Mass spectrometer 630 is controlled by processor 640, for example.

In the system of FIG. 6, mass spectrometer 630 is shown as a triple quadrupole device. One of ordinary skill in the art can appreciate that any component of mass spectrometer 630 can include other types of mass spectrometry devices including, but not limited to, ion traps, orbitraps, TOF devices, ion mobility devices, or Fourier transform ion cyclotron resonance FT-ICR devices.

In various embodiments, the system of FIG. 6 further includes additional device 610 that affects compound 601, providing the at least one additional dimension. As shown in FIG. 6, additional device 610 is an LC device and the at least one additional dimension or spectral data provided is retention time. In various alternative embodiments, additional device 610 can be, but is not limited to, a GC device, a CE device, an IMS device, or a DMS device. In still further embodiments, additional device 610 is not used and the at least one additional dimension or spectral data provided is precursor ion m/z and is provided by mass spectrometer 630 operating in a precursor ion scanning mode.

### Method for Automatically Determining a Mass Tolerance

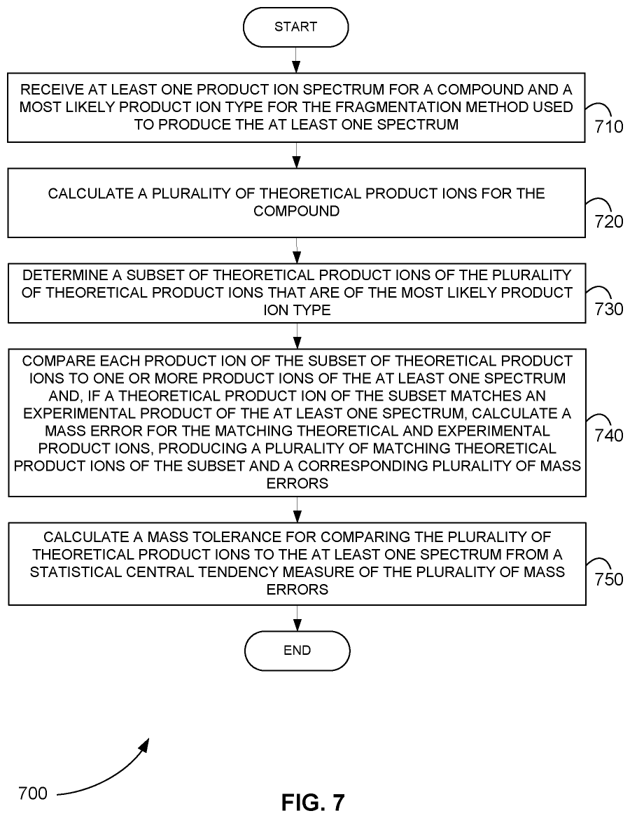

FIG. 7 is an exemplary flowchart showing a method 700 for automatically determining a mass tolerance for comparing theoretical mass peaks to mass peaks of an experimental mass spectrum, in accordance with various embodiments.

In step 710 of method 700, at least one product ion spectrum for a compound and a most likely product ion type (e.g. b or y or c′ or z^{⋅}) and the sequence for the fragmentation method used to produce the at least one spectrum are received.

In step 720, a plurality of theoretical product ions is calculated for the compound.

In step 730, a subset of theoretical product ions of the plurality of theoretical product ions that are of the most likely product ion type is determined.

In step 740, each product ion of the subset of theoretical product ions is compared to one or more product ions of the at least one spectrum and, if a theoretical product ion of the subset matches an experimental product of the at least one spectrum, a mass error is calculated for the matching theoretical and experimental product ions. A plurality of matching theoretical product ions of the subset and a corresponding plurality of mass errors are produced.

In various embodiments, a predetermined mass tolerance can be used to compare each product ion of the subset of theoretical product ions to one or more product ions of spectrum 631. In various embodiments, isotopes can be used to compare each product ion of the subset of theoretical product ions to one or more product ions of spectrum 631. In that case, both a predetermined mass tolerance and intensity can be used. Also, in various embodiments, possible matches (within mass tolerance) can be removed based on other criteria such as isotope pattern match.

In step 750, a mass tolerance for comparing the plurality of theoretical product ions to the at least one spectrum is calculated from a statistical central tendency measure of the plurality of mass errors.

### Computer Program Product for Automatically Determining a Mass Tolerance

In various embodiments, a computer program product includes a non-transitory tangible computer-readable storage medium whose contents include a program with instructions being executed on a processor so as to perform a method for automatically determining a mass tolerance for comparing theoretical mass peaks to mass peaks of an experimental mass spectrum. This method is performed by a system that includes one or more distinct software modules.

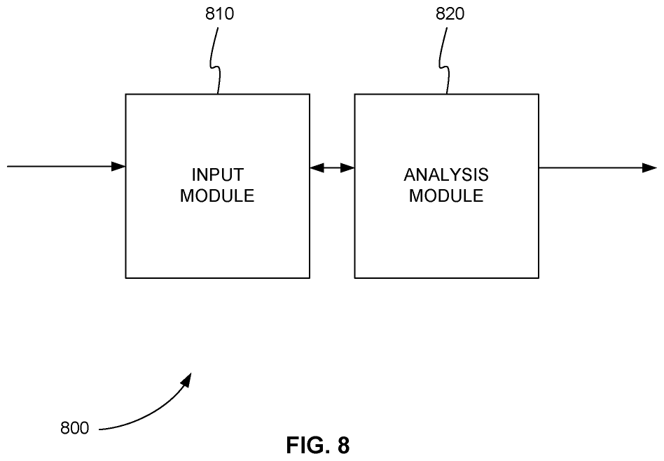

FIG. 8 is a schematic diagram of a system 800 that includes one or more distinct software modules and that performs a method for automatically determining a mass tolerance for comparing theoretical mass peaks to mass peaks of an experimental mass spectrum, in accordance with various embodiments. System 800 includes input module 810 and analysis module 820.

Input module 810 receives at least one product ion spectrum for a compound and a most likely product ion type for the fragmentation method used to produce the at least one spectrum.

Analysis module 820 calculates a plurality of theoretical product ions for the compound. Analysis module 820 determines a subset of theoretical product ions of the plurality of theoretical product ions that are of the most likely product ion type.

Analysis module 820 compares each product ion of the subset of theoretical product ions to one or more product ions of the at least one spectrum and, if a theoretical product ion of the subset matches an experimental product of the at least one spectrum, calculates a mass error for the matching theoretical and experimental product ions. A plurality of matching theoretical product ions of the subset and a corresponding plurality of mass errors are produced.

Analysis module 820 calculates a mass tolerance for comparing the plurality of theoretical product ions to the at least one spectrum from a statistical central tendency measure of the plurality of mass errors.

### System for Finding a Product Ion Using Charge States

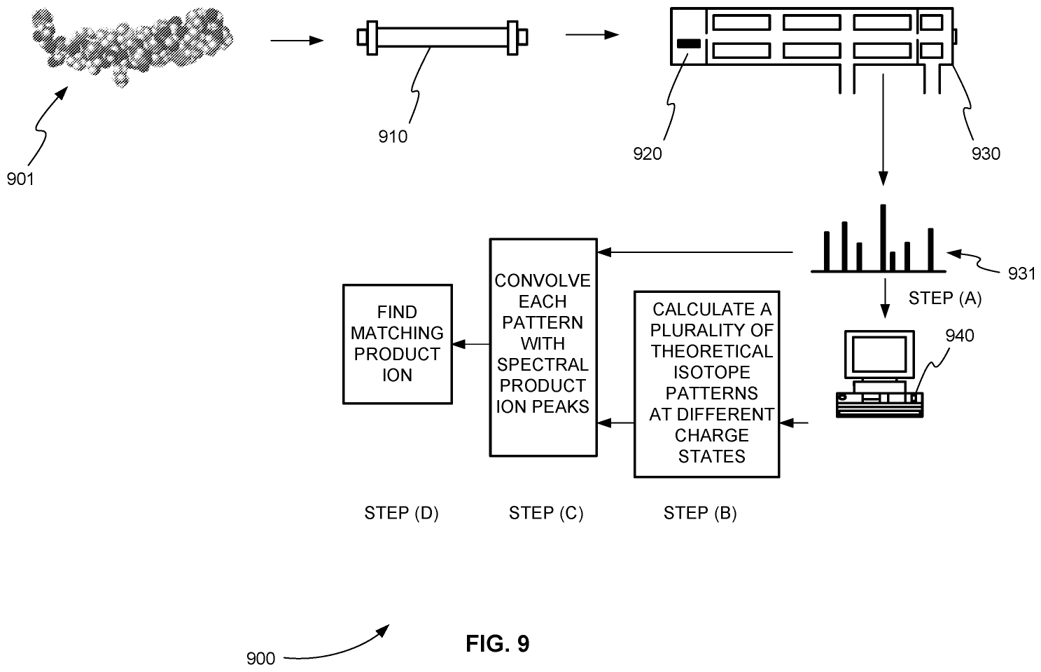

FIG. 9 is a schematic diagram 900 of a system for finding an experimental product ion of a compound using charge states, in accordance with various embodiments. The system includes processor 940. Processor 940 can be, but is not limited to, a controller, a computer, a microprocessor, the computer system of FIG. 1, or any device capable of analyzing data. Processor 940 can also be any device capable of sending and receiving control signals and data.

In a step (A), processor 940 receives at least one product ion spectrum 931 of compound 901. In step (B), processor 940 calculates for at least one theoretical product ion of compound 901 a plurality of theoretical isotope patterns corresponding to different possible charge states of the theoretical product ion.

In step (C), processor 940 convolves each pattern of the plurality of theoretical isotope patterns with product ion peaks of spectrum 931.

In step (D), if a theoretical isotope pattern of the plurality of theoretical isotope patterns aligns with a number of peaks of a product ion of the at least one spectrum that is greater than a predetermined threshold number, processor 940 finds that the product ion matches the at least one theoretical product ion of the compound.

In various embodiments, compound 901 includes a polymeric compound and the at least one theoretical product ion is calculated for a bond of the polymeric compound.

In various embodiments, steps (B)-(D) are performed after the at least one theoretical product ion is compared to spectrum 931 by another method and no match is found.

In various embodiments, the charge of the product ion matching the at least one theoretical product ion of a compound is taken as the charge of the matching theoretical isotope pattern with the highest convolution score.

In various embodiments, the system of FIG. 9 further includes mass spectrometer 930 that measures mass spectrum 931 and sends mass spectrum 931 to processor 930. Ion source device 920 of mass spectrometer 930 ionizes separated fragments of compound 901 or only compound 901, producing an ion beam. Ion source device 920 is controlled by processor 940, for example. Ion source device 920 is shown as a component of mass spectrometer 930. In various alternative embodiments, ion source device 920 is a separate device. Ion source device 920 can be, but is not limited to, an ESI device or a CI source device such as an APCI device or an APPI source device.

Mass spectrometer 930 mass analyzes product ions of compound 901 or selects and fragments compound 901 and mass analyzes product ions of compound 901 from the ion beam at a plurality of different times. Mass spectrum 931 is produced for compound 901. Mass spectrometer 930 is controlled by processor 940, for example.

In the system of FIG. 9, mass spectrometer 930 is shown as a triple quadrupole device. One of ordinary skill in the art can appreciate that any component of mass spectrometer 930 can include other types of mass spectrometry devices including, but not limited to, ion traps, orbitraps, TOF devices, ion mobility devices, or Fourier transform ion cyclotron resonance FT-ICR devices.

In various embodiments, the system of FIG. 9 further includes additional device 910 that affects compound 901, providing the at least one additional dimension. As shown in FIG. 9, additional device 910 is an LC device and the at least one additional dimension or spectral data provided is retention time. In various alternative embodiments, additional device 910 can be, but is not limited to, a GC device, a CE device, an IMS device, or a DMS device. In still further embodiments, additional device 910 is not used and the at least one additional dimension or spectral data provided is precursor ion m/z and is provided by mass spectrometer 930 operating in a precursor ion scanning mode.

### Method for Finding a Product Ion Using Charge States

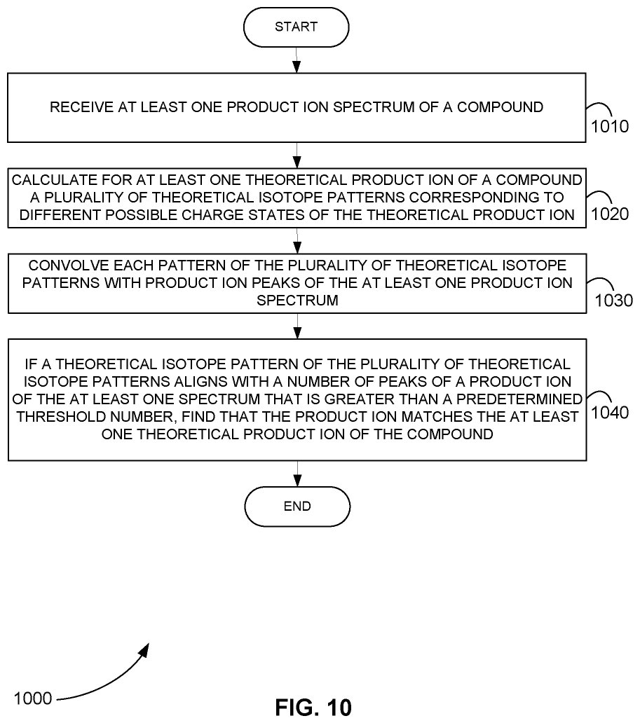

FIG. 10 is an exemplary flowchart showing a method 1000 for finding an experimental product ion of a compound using charge states, in accordance with various embodiments.

In step 1010 of method 1000, at least one product ion spectrum of a compound is received.

In step 1020, for at least one theoretical product ion of a compound, a plurality of theoretical isotope patterns corresponding to different possible charge states of the theoretical product ion is calculated.

In step 1030, each pattern of the plurality of theoretical isotope patterns is convolved with product ion peaks of the at least one product ion spectrum

In step 1040, if a theoretical isotope pattern of the plurality of theoretical isotope patterns aligns with a number of peaks of a product ion of the at least one spectrum that is greater than a predetermined threshold number, the product ion matches the at least one theoretical product ion of the compound.

### Computer Program Product for Finding a Product Ion Using Charge States

In various embodiments, a computer program product includes a non-transitory tangible computer-readable storage medium whose contents include a program with instructions being executed on a processor so as to perform a method for finding an experimental product ion of a compound using charge states. This method is performed by a system that includes one or more distinct software modules.

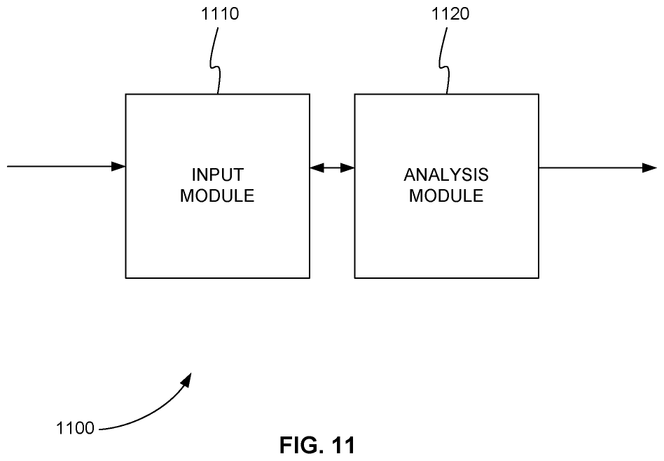

FIG. 11 is a schematic diagram of a system 1100 that includes one or more distinct software modules and that performs a method for finding an experimental product ion of a compound using charge states, in accordance with various embodiments. System 1100 includes input module 1110 and analysis module 1120.

Input module 1110 receives at least one product ion spectrum of a compound.

Analysis module 1120 calculates for at least one theoretical product ion of the compound a plurality of theoretical isotope patterns corresponding to different possible charge states of the theoretical product ion. Analysis module 1120 convolves each pattern of the plurality of theoretical isotope patterns with product ion peaks of the at least one product ion spectrum.

If a theoretical isotope pattern of the plurality of theoretical isotope patterns aligns with a number of peaks of a product ion of the at least one spectrum that is greater than a predetermined threshold number, analysis module 1120 finds that the product ion matches the at least one theoretical product ion of the compound

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

What is claimed is:

1. A method for determining if internal product ions are used to provide evidence for a bond of a polymeric compound, comprising:
(a) receiving a sequence and at least one product ion spectrum for a polymeric compound;
(b) calculating two or more theoretical product ions resulting from the cleavage of at least one bond of the sequence;
(c) searching the at least one product ion spectrum for the two or more theoretical product ions; and
(d) only if one or more theoretical product ions of the two or more theoretical product ions match a product ion of the at least one product ion spectrum, calculating theoretical internal product ions for the one or more matching theoretical product ions and searching the at least one product ion spectrum for theoretical internal product ions of the one or more theoretical product ions.

2. The method of claim 1, further comprising in step (d)
if the one or more theoretical product ions of the two or more theoretical product ions are one or more of b, y, c′, or z^{•} product ions and match a product ion of the at least one product ion spectrum, calculating theoretical internal product ions for the one or more b, y, c′, or z^{•} theoretical product ions and searching the at least one product ion spectrum for theoretical internal product ions of the one or more b, y, c′, or z^{•} theoretical product ions.

3. The method of claim 1, further comprising in step (d)
if all of the two or more theoretical product ions match a product ion of the at least one product ion spectrum, calculating theoretical internal product ions for the two or more theoretical product ions and searching the at least one product ion spectrum for theoretical internal product ions of the two or more theoretical product ions.

4. (canceled)

5. A system for determining if internal product ions are used to provide evidence for a bond of a polymeric compound, comprising:
a processor that
receives a sequence and at least one product ion spectrum for a polymeric compound,
calculates two or more theoretical product ions resulting from the cleavage of at least one bond of the sequence,
searches the at least one product ion spectrum for the two or more theoretical product ions, and
only if one or more theoretical product ions of the two or more theoretical product ions match a product ion of the at least one product ion spectrum, calculates theoretical internal product ions for the one or more theoretical product ions and searches the at least one product ion spectrum for theoretical internal product ions of the one or more theoretical product ions.

6-10. (canceled)

11. A method for finding an experimental product ion of a compound using charge states, comprising:
(a) receiving at least one product ion spectrum of a compound;
(b) calculating for at least one theoretical product ion of the compound a plurality of theoretical isotope patterns corresponding to different possible charge states of the theoretical product ion;
(c) convolving each pattern of the plurality of theoretical isotope patterns with product ion peaks of the at least one product ion spectrum;
(d) if a theoretical isotope pattern of the plurality of theoretical isotope patterns aligns with a number of peaks of a product ion of the at least one product ion spectrum that is greater than a predetermined threshold number, finding that the product ion matches the at least one theoretical product ion of the compound.

12. The method of claim 11, wherein the compound comprises a polymeric compound and the at least one theoretical product ion is calculated for a broken bond of the polymeric compound.

13. The method of claim 11, wherein steps (b)-(d) are performed after the at least one theoretical product ion is compared to the at least one product ion spectrum by another method and no match is found.

14. The method of claim 11, wherein a charge of the product ion matching the at least one theoretical product ion of a compound is taken as a charge of the matching theoretical isotope pattern with a highest convolution score.

15-16. (canceled)
